(19) United States
(12) Patent Application Publication
(10) Pub. No.: US 2026/0061089 A1
(43) Pub. Date: Mar. 5, 2026

(54) FRAGRANCE DIFFUSION EQUIPMENT

(71) Applicant: GUANGDONG CHIYANG SCENT TECHNOLOGY CO., LTD, Foshan (CN)

(72) Inventor: Xiaoyang GAO, Foshan (CN)

(73) Assignee: GUANGDONG CHIYANG SCENT TECHNOLOGY CO., LTD, Foshan (CN)

(21) Appl. No.: 18/955,596

(22) Filed: Nov. 21, 2024

(30) Foreign Application Priority Data
Sep. 4, 2024 (CN) 2024221606415

(51) Int. Cl. A61L 9/14

(52) U.S. Cl. CPC A61L 9/14; A61L 2209/133; A61L 2209/134

(57) ABSTRACT

The present invention provides fragrance diffusion equipment, including an atomizing head, a liquid storage bottle, and an air pump, the atomizing head being connected to the liquid storage bottle, where the atomizing head includes an atomizing head body, an atomizing core, a liquid suction pipe, and a liquid collecting tank, the atomizing head body is connected to the liquid storage bottle, the atomizing core is connected to the atomizing head body, one end on the liquid suction pipe is connected to the atomizing core, the other end of the liquid suction pipe is inserted into the liquid storage bottle, the air pump is in communication with the atomizing core; and the liquid collecting tank is connected to a bottom end of the atomizing head body, the atomizing core is enclosed between the atomizing head body and the liquid collecting tank.

### CROSS-REFERENCE TO RELATED APPLICATIONS

The application claims priority to Chinese patent application No. 2024221606415, filed on Sep. 4, 2024, the entire contents of which are incorporated herein by reference.

### TECHNICAL FIELD

The present invention relates to the technical field of atomization devices, and in particular, to fragrance diffusion equipment.

### BACKGROUND

With the continuous improvement of living standards and the continuous pursuit of life quality, in order to improve the air environment, people mostly use a method of spraying perfume or essential oil and other fresheners indoors to improve the air quality, so as to achieve the effects of eliminating peculiar smell, keeping air freshening and sterilizing and disinfecting. However, according to the method, the fragrance can be kept only by spraying the freshener to the air frequently, which is inconvenient to use, and since the sprayed freshener particles are relatively large, the floating range is relatively small, and therefore, this method is low in efficiency and large in waste. On the basis of this, people invented a fragrance diffusion equipment. The fragrance diffusion equipment atomizes perfume or essential oil and other fresheners at an atomizer through the negative pressure principle, and the freshener is discharged from a mist outlet, so that the fragrance is diffused into the air to improve the air quality.

Because the atomizing channel of most existing fragrance diffusion equipment is in communication with the interior of the essential oil bottle from the mist outlet, a large amount of oil liquid in the essential oil bottle can be leaked from the atomizing channel after the fragrance diffusion equipment being toppled over accidentally. The large amount of liquid leakage not only causes waste, but also enables the oil liquid to enter an electric control assembly of the fragrance diffusion equipment to corrode a circuit board or damage an air pump, causing damage to the equipment, and in severe cases, a fire disaster may even be caused due to a short circuit.

### SUMMARY

In view of this, the present invention provides fragrance diffusion equipment, so as to solve the problem that a large amount of liquid leakage occurs after the fragrance diffusion equipment being toppled over accidentally.

In order to solve the above technical problems, the present invention adopts the following technical solution: fragrance diffusion equipment, including an atomizing head, a liquid storage bottle, and an air pump, the atomizing head being connected to the liquid storage bottle, where the atomizing head includes an atomizing head body, an atomizing core, a liquid suction pipe, and a liquid collecting tank, the atomizing head body is connected to the liquid storage bottle, the atomizing core is connected to the atomizing head body, one end on the liquid suction pipe is connected to the atomizing core, the other end of the liquid suction pipe is inserted into the liquid storage bottle, the air pump is in communication with the atomizing core and is used for providing air flow to the atomizing core, so that the atomizing core atomizes the liquid sucked by the liquid suction pipe; and

the liquid collecting tank is connected to a bottom end of the atomizing head body, the atomizing core is enclosed between the atomizing head body and the liquid collecting tank, the atomizing core and the liquid collecting tank extend into a bottle mouth of the liquid storage bottle, and a liquid return hole communicated with the liquid storage bottle is further formed in the liquid collecting tank.

Preferably, the liquid return hole is formed by a gap that is formed between the liquid collecting tank and a bottom end of the atomizing core after penetrating through the liquid collecting tank;

and/or, a via hole communicated with the liquid storage bottle is disposed on the liquid collecting tank, and the via hole is higher than an inner bottom of the liquid collecting tank.

Further preferably, the bottom end of the atomizing core penetrates through the liquid collecting tank and then extends into the liquid storage bottle, one end of the liquid suction pipe is in communication with the bottom end of the atomizing core, and the other end of the liquid suction pipe is inserted into the liquid storage bottle.

Further preferably, a radial length of the gap ranges from 0.20 mm to 0.40 mm;
and/or, a side wall of the liquid collecting tank is provided with a first columnar wall recessed inwards, a top side of the first columnar wall is provided with a first top plate, and the via hole is formed in the first top plate;
and/or, a diameter of via hole ranges from 0.5 mm to 1.0 mm.

Preferably, the liquid collecting tank is detachably connected to the atomizing head body, and a top edge of the liquid collecting tank is tightly matched with the atomizing head body.

Further preferably, the side wall of the liquid collecting tank is provided with a second columnar wall recessed inwards, a second top plate is disposed on a top side of the second columnar wall, the liquid collecting tank is fixedly connected to the atomizing head body through a bolt which penetrates from the second top plate and then is inserted into the atomizing head body, and the top edge of the liquid collecting tank is tightly matched with the atomizing head body.

Further preferably, an atomizing cavity is disposed in the atomizing head body, a first atomizing cover is disposed in the atomizing cavity, the first atomizing cover divides the atomizing cavity into an upper cavity and a lower cavity which are in communication with each other, and the atomizing core and the liquid collecting tank are both located in the lower cavity.

Further preferably, a second atomizing cover is further disposed in the lower cavity, a first atomizing channel is disposed at intervals in a circumferential direction of the first atomizing cover, the first atomizing channel is in communication with the upper cavity and the lower cavity, the second atomizing cover is located between the first atomizing cover and the atomizing core, and a second atomizing channel in communication with the lower cavity is formed between the second atomizing cover and the first atomizing cover.

Further preferably, the atomizing head body is provided with a gas channel in communication with the atomizing core, the gas channel is located below the second atomizing cover, a support portion is disposed at the bottom of the second atomizing cover, and the support portion abuts against a top side of the gas channel.

Further preferably, the second atomizing cover includes a cover plate protruding towards the first atomizing cover, a vertical surrounding plate is disposed on the periphery of the cover plate, and a backflow gap is disposed between an outer side of the surrounding plate and the first atomizing cover; and

the first atomizing cover is provided with several pressing blocks at intervals along the periphery of a top side of the surrounding plate, the pressing blocks are matched with the support portion to vertically fix the second atomizing cover; and/or, the surrounding plate is provided with a positioning groove, the first atomizing cover is provided with a positioning plate, and the positioning plate is inserted into the positioning groove to circumferentially fix the second atomizing cover.

Compared with the prior art, beneficial effects of the fragrance diffusion equipment disclosed by the present invention are as follows:

In the present invention, the liquid collecting tank is disposed, so that the atomizing core is enclosed between the atomizing head body and the liquid collecting tank, and the liquid collecting tank extends into the bottle mouth of the liquid storage bottle and then is connected to the atomizing head body, the bottle mouth of the liquid storage bottle can be blocked to the maximum extent under the condition that the smoothness of the atomizing channel of the fragrance diffusion equipment is not affected, so that a large amount of liquid in the bottle from suddenly flowing out when the fragrance diffusion equipment is toppled over is prevented, time is striven for a user to find the pouring condition in time, the loss of a large amount of liquid leakage is avoided, and the user experience is improved.

### BRIEF DESCRIPTION OF THE DRAWINGS

FIG. 1 is a perspective view of a part of a structure of fragrance diffusion equipment provided by an embodiment of the present invention;

FIG. 2 is a perspective view 1 of an internal structure of FIG. 1;

FIG. 3 is a perspective view 2 of an internal structure of FIG. 1;

FIG. 4 is a three-dimensional structural diagram of the liquid collecting tank provided by an embodiment of the present invention; and

FIG. 5 is a three-dimensional structural diagram of the second atomizing cover provided by an embodiment of the present invention.

Reference numerals in the accompanying drawings: atomizing head 1, liquid collecting tank 2, liquid storage bottle 3, atomizing core 4, liquid return hole 5, via hole 6, first columnar wall 7, second columnar wall 8, first top plate 9, second top plate 10, first atomizing cover 11, pressing block 110, positioning plate 111, upper cavity 12, lower cavity 13, second atomizing cover 14, cover plate 140, surrounding plate 141, positioning groove 142, support portion 143, first atomizing channel 15, second atomizing channel 16, gas channel 17, mist outlet 18, atomizing head body 19, liquid suction pipe 20.

### DETAILED DESCRIPTION OF THE EMBODIMENTS

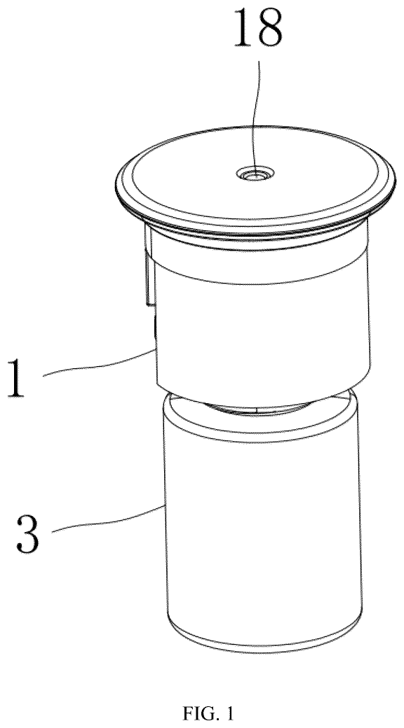

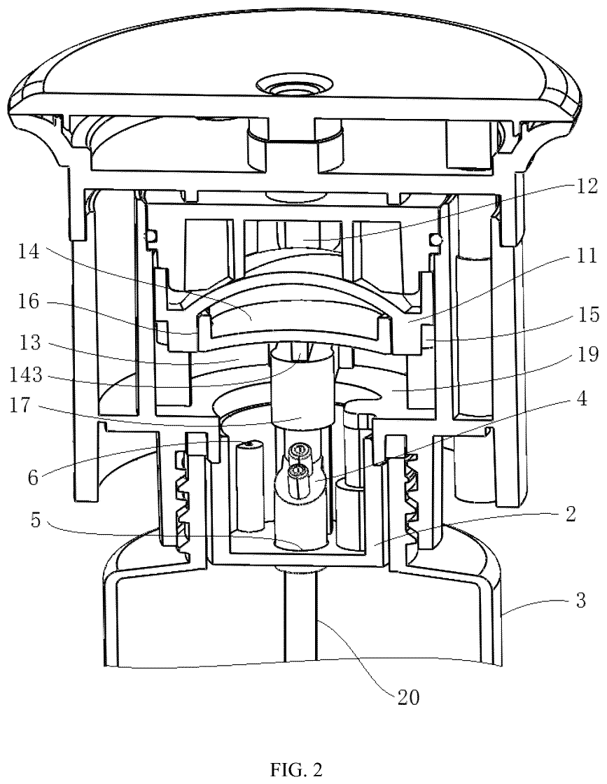

This embodiment provides fragrance diffusion equipment, as shown in FIG. 1 and FIG. 2, including an atomizing head 1, a liquid storage bottle 3, and an air pump (not shown in the figure), the atomizing head 1 being connected to the liquid storage bottle 3, where the atomizing head 1 includes an atomizing head body 19, an atomizing core 4, a liquid suction pipe 20, and a liquid collecting tank 2, the atomizing head body 19 is connected to the liquid storage bottle 3, the atomizing core 4 is connected to the atomizing head body 19, one end on the liquid suction pipe 20 is connected to the atomizing core 4, the other end of the liquid suction pipe is inserted into the liquid storage bottle 3, the air pump is in communication with the atomizing core 4 and is used for providing air flow to the atomizing core 4, so that the atomizing core 4 atomizes the liquid sucked by the liquid suction pipe 20; and the liquid collecting tank 2 is connected to a bottom end of the atomizing head body 19, the atomizing core 4 is enclosed between the atomizing head body 19 and the liquid collecting tank 2, the atomizing core 4 and the liquid collecting tank 2 extend into a bottle mouth of the liquid storage bottle 3, and a liquid return hole communicated with the liquid storage bottle 3 is further formed in the liquid collecting tank 2.

In the present invention, the liquid collecting tank 2 is disposed, so that the atomizing core 4 is enclosed between the atomizing head body 19 and the liquid collecting tank 2, and the liquid collecting tank 2 extends into the bottle mouth of the liquid storage bottle 3 and then is connected to the atomizing head body 19, the bottle mouth of the liquid storage bottle 3 can be blocked to the maximum extent under the condition that the smoothness of the atomizing channel of the fragrance diffusion equipment is not affected, so that a large amount of liquid in the bottle from suddenly flowing out when the fragrance diffusion equipment is toppled over is prevented, time is striven for a user to find the pouring condition in time, the loss of a large amount of liquid leakage is avoided, and the user experience is improved.

In a preferred embodiment, the liquid return hole 5 is formed by a gap that is formed between the liquid collecting tank 2 and a bottom end of the atomizing core 4 after penetrating through the liquid collecting tank 2. It should be noted that the gap can be continuously disposed in a circumferential direction, or may be discontinuously disposed. The liquid return hole 5 is used for enabling the oil liquid falling back due to the excessively large atomized particles to flow back into the liquid storage bottle 3 through the liquid return hole 5 during the atomization operation of the fragrance diffusion equipment.

In a preferred embodiment, in order to prevent the oil liquid in the bottle from continuously flowing out from the liquid return hole 5 after the liquid storage bottle 3 being toppled over, the liquid return hole 5 in communication with the liquid storage bottle 3 disposed on the liquid collecting tank 2 is used, and the diameter of the liquid return hole 5 is set to a certain numerical value or numerical range. Because when the diameter of the liquid return hole 5 is small to a certain extent, a state in which a liquid channel and a gas channel 17 coexist cannot be formed in the liquid return hole 5. In addition, by using the characteristic that essential oil has a certain viscosity, the oil liquid is prone to form certain surface tension in the liquid return hole 5, so that the liquid in the liquid storage bottle 3 cannot continuously flow out of the liquid return hole 5 after the liquid storage bottle 3 being toppled over, thereby avoiding the problem of continuous oil leakage and further reducing liquid leakage loss.

It should be noted that the liquid storage bottle 3 is toppled over when the equipment is not started, liquid in the liquid storage bottle 3 can flood the liquid return hole 5, which may cause the liquid to flow out for a short time. However, after the liquid flows out of a little bit, a pressure difference is generated between two ends of the liquid return hole 5, that is, between inside and outside of the liquid storage bottle 3, and when a certain pressure difference is generated between the inside and the outside of the liquid storage bottle 3, the liquid in the bottle cannot continuously flow out. When the liquid storage bottle 3 is toppled over during the atomization operation of the equipment, because the atomizing head 1 continuously sprays liquid by means of a negative pressure principle, the interior of the liquid storage bottle 3 becomes a continuous negative pressure cavity, and in this way, the liquid in the liquid storage bottle 3 cannot continuously flow out from the liquid return hole 5, and naturally, liquid leakage is impossible.

Specifically, the bottom end of the atomizing core 4 penetrates through the liquid collecting tank 2 and then extends into the liquid storage bottle 3, one end of the liquid suction pipe 20 is in communication with the bottom end of the atomizing core 4, and the other end of the liquid suction pipe is inserted into the liquid storage bottle 3.

In a preferred embodiment, in order to ensure that liquid leakage is not prone to occurring when the liquid storage bottle 3 is toppled over, and to enable the liquid return hole to enable the liquid converged in the liquid collecting tank 2 to flow back into the liquid storage bottle 3 during normal (that is, the liquid storage bottle 3 is in an upright state) atomization operation of the equipment, the radial length of the liquid return hole 5 ranges from 0.20 mm to 0.40 mm, preferably 0.30 mm, that is, a unilateral radial gap between the periphery of the atomizing core 4 and the bottom plate of the liquid collecting tank 2 is 0.30 mm. It can be understood that when the value is reduced based on 0.30 mm, the problem of liquid leakage due to toppling is not prone to occurring, or time for maintaining the non-continuous liquid leakage is longer, but at the same time, the backflow smoothness of the liquid in the liquid collecting tank 2 during the normal atomization operation is also reduced. Similarly, when the value is increased based on 0.30 mm, the problem of liquid leakage due to toppling is relatively easy to occur, or time for maintaining the non-continuous liquid leakage is shortened, but at the same time, the backflow smoothness of the liquid in the liquid collecting tank 2 during the normal atomization operation is also improved.

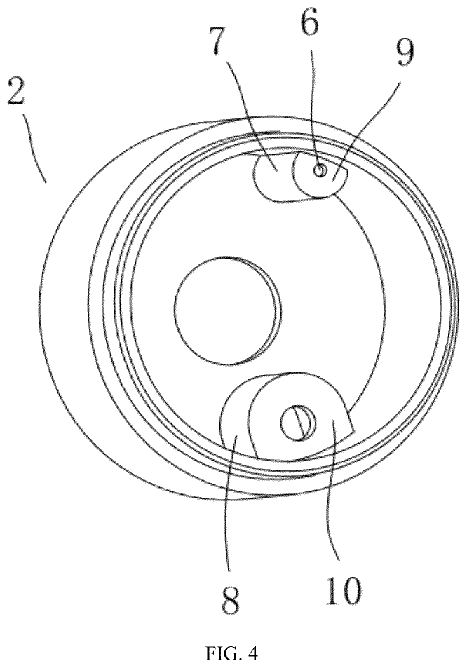

In another preferred embodiment, as shown in FIG. 2 and FIG. 4, a via hole 6 communicated with the liquid storage bottle 3 is disposed on the liquid collecting tank 2, and the via hole 6 is higher than an inner bottom of the liquid collecting tank 2. In this embodiment, the via hole 6 is disposed, so that the other channel other than the atomizing core 4 and the liquid return hole 5 is provided for internal and external conduction of the liquid storage bottle 3, the internal and external air pressure balance of the liquid storage bottle 3 can be promoted, liquid in the liquid collecting tank 2 is promoted to smoothly flow back into the liquid storage bottle 3 from the liquid return hole 5, and the backflow speed at the liquid return hole 5 is ensured. The via hole 6 is arranged to be higher than the inner bottom of the liquid collecting tank 2, so that the via hole 6 is prevented from being flooded by the liquid converged at the inner bottom of the liquid collecting tank 2 during the atomization operation of the equipment. It should be noted that a distance between the via hole 6 and the inner bottom of the liquid collecting tank 2 can be determined comprehensively based on the atomization amount of the equipment, the size of the atomized particles, the operation time of a single atomization, and the like. Under the condition that other equipment structures and test conditions are the same, according to the embodiment, the equipment is toppled over during non-operation, the duration of maintaining the non-leaking liquid for equipment is shortened to a certain extent relative to the condition that the via hole 6 is not disposed. The size of the via hole 6 and the liquid return hole 5 can be correspondingly debugged and determined according to the specific requirements of the duration of maintaining the non-leaking liquid.

Therefore, in a further preferred embodiment, in order to ensure that liquid leakage is not prone to occurring during non-operation of the equipment under the condition that the via hole 6 is disposed, the diameter of the via hole 6 preferably ranges from 0.5 mm to 1.0 mm. According to the embodiment, the backflow speed at the liquid return hole 5 and the function that liquid leakage is not prone to occurring after toppling can be considered. In this embodiment, the test results show that the horizontal toppling (the toppling angle is 90 degrees) of the liquid storage bottle 3 is not easy to leak oil for 10 minutes, and the vertical toppling (the toppling angle is 180 degrees) of the liquid storage bottle is not easy to leak oil for 2 minutes. It should be noted that when the liquid storage bottle 3 is toppled over during the atomization operation of the equipment, because the atomizing head 1 continuously sprays liquid by means of a negative pressure principle, the interior of the liquid storage bottle 3 becomes a continuous negative pressure cavity, in this way, the liquid in the liquid storage bottle 3 cannot continuously flow out from the via hole 6, and the liquid leakage function is basically not affected. When the equipment is in normal (that is, the liquid storage bottle 3 is in an upright state) atomization operation, if atomized particles fall into the via hole 6 to block the via hole 6, the liquid in the via hole 6 can quickly flow back into the liquid storage bottle 3 due to the negative pressure cavity maintained inside the liquid storage bottle 3, so that the via hole 6 is rapidly restored to be unblocked.

As an example, in a preferred embodiment, as shown in FIG. 4, a side wall of the liquid collecting tank 2 is provided with a first columnar wall 7 recessed inwards, a top side of the first columnar wall 7 is provided with a first top plate 9, and the via hole 6 is formed in the first top plate 9. In this way, on one hand, the side wall of the liquid collecting tank 2 serves as a surrounding wall of the hole, materials can be saved, and it is convenient for integral injection molding, and meanwhile the stability of the structure of the via hole 6 can be ensured.

In a preferred embodiment, the liquid collecting tank 2 and the atomizing head body 19 are detachably connected, such as a bolt connection, so as to facilitate structural design and mounting of the atomizing head body 19. In addition, the top edge of the liquid collecting tank 2 is tightly matched with the atomizing head body 19, so as to prevent the liquid in the liquid storage bottle 3 from leaking out of the connection position. Further, the side wall of the liquid collecting tank 2 is provided with a second columnar wall 8 recessed inwards, a second top plate 10 is disposed on a top side of the second columnar wall 8, the liquid collecting tank 2 is fixedly connected to the atomizing head body 19 through a bolt which penetrates from the second top plate 10 and then is inserted into the atomizing head body 19, and the top edge of the liquid collecting tank 2 is tightly matched with the atomizing head body 19. In this way, during mounting, the liquid channel structure of the atomizing core 4 only needs to penetrate through a preset through hole at an inner bottom of the liquid collecting tank 2, a bolt hole in the second top plate 10 is aligned with a bolt hole corresponding to the atomizing head body 19, the bolt is inserted and tightened, and the mounting of the liquid collecting tank 2 and the atomizing head body 19 can be completed, which is convenient and quick.

In a preferred embodiment, as shown in FIG. 2, an atomizing cavity is disposed in the atomizing head body 19, a first atomizing cover 11 is disposed in the atomizing cavity, the first atomizing cover 11 divides the atomizing cavity into an upper cavity 12 and a lower cavity 13 which are in communication with each other, and the atomizing core 4 and the liquid collecting tank 2 are both located in the lower cavity 13. The first atomizing cover 11 is a conventional arrangement in the art, and functions of the first atomizing cover in reducing noise, reducing atomized particles of the mist outlet 18 and promoting backflow of the larger atomized particles are not repeated here.

In a preferred embodiment, a second atomizing cover 14 is further disposed in the lower cavity 13, a first atomizing channel 15 is disposed at intervals in a circumferential direction of the first atomizing cover 11, the first atomizing channel 15 is in communication with the upper cavity 12 and the lower cavity 13, the second atomizing cover 14 is located between the first atomizing cover 11 and the atomizing core 4, and a second atomizing channel 16 in communication with the lower cavity 13 is formed between the second atomizing cover 14 and the first atomizing cover 11. In this embodiment, the second atomizing cover 14 is disposed, so that the atomized particles can enter the first atomizing channel 15 and then continue to blow out to the mist outlet 18, a large amount of atomized particles are prevented from directly dispersing into the first atomizing cover 11 and adhering to the first atomizing cover 11 to flow back, and then the atomization amount can be improved; and the second atomizing channel 16 may allow larger atomization particles that blow into the cavity between the first atomizing cover 11 and the second atomizing cover 14 to flow back.

Further, the atomizing head body 19 is provided with a gas channel 17 in communication with the atomizing core 4, the gas channel 17 is located below the second atomizing cover 14, and a support portion 143 is disposed at the bottom of the second atomizing cover 14. In the embodiment, the support portion 143 is a vertical support plate, the support plate abuts against a top side of the gas channel 17, and the second atomizing cover 14 is fixed through abutting of the support plate and the gas channel 17.

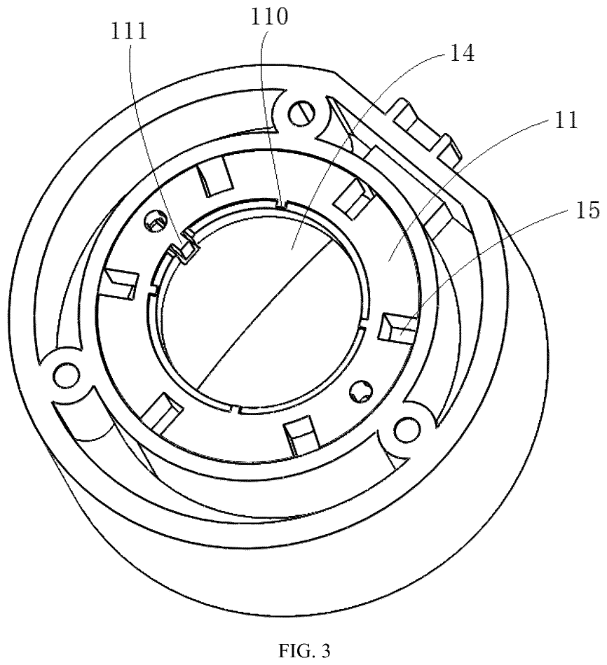

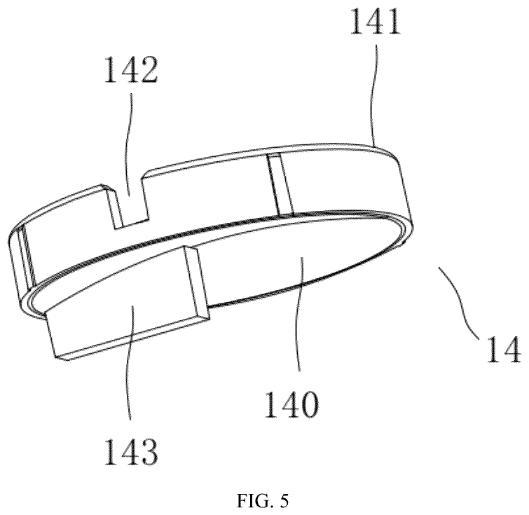

In a specific embodiment, as shown in FIG. 5, the second atomizing cover 14 includes a cover plate 140 protruding towards the first atomizing cover 11, facilitating backflow of the atomization particles; and a vertical surrounding plate 141 is disposed on the periphery of the cover plate 140, a backflow gap is disposed between an outer side of the surrounding plate 141 and the first atomizing cover 11, and the backflow gap is a component of the second atomizing channel 16;
as shown in FIG. 3, the first atomizing cover 11 is provided with several pressing blocks 110 at intervals along the periphery of a top side of the surrounding plate 141, and the pressing blocks 110 are matched with the support portion 143 to vertically fix the second atomizing cover 14, so that the second atomizing cover 14 does not move up and down; and
the surrounding plate 141 is provided with a positioning groove 142, the first atomizing cover 11 is provided with a positioning plate 111, and the positioning plate 111 is inserted into the positioning groove 142 to circumferentially fix the second atomizing cover 14, so that the second atomizing cover 14 does not rotate.

Although the embodiments of this application are shown and described above, it can be understood that the foregoing embodiments are examples and shall not be construed as a limitation on this application. A person of ordinary skill in the art may make changes, modifications, substitutions, and variants based on the foregoing embodiments within the scope of this application.

What is claimed is:

1. Fragrance diffusion equipment, comprising an atomizing head, a liquid storage bottle, and an air pump, the atomizing head being connected to the liquid storage bottle, wherein the atomizing head comprises an atomizing head body, an atomizing core, a liquid suction pipe, and a liquid collecting tank, the atomizing head body is connected to the liquid storage bottle, the atomizing core is connected to the atomizing head body, one end on the liquid suction pipe is connected to the atomizing core, the other end of the liquid suction pipe is inserted into the liquid storage bottle, the air pump is in communication with the atomizing core and is used for providing air flow to the atomizing core, so that the atomizing core atomizes the liquid sucked by the liquid suction pipe; and
the liquid collecting tank is connected to a bottom end of the atomizing head body, the atomizing core is enclosed between the atomizing head body and the liquid collecting tank, the atomizing core and the liquid collecting tank extend into a bottle mouth of the liquid storage bottle, and a liquid return hole communicated with the liquid storage bottle is further formed in the liquid collecting tank.

2. The fragrance diffusion equipment according to claim 1, wherein the liquid return hole is formed by a gap that is formed between the liquid collecting tank and a bottom end of the atomizing core after penetrating through the liquid collecting tank;
and/or, a via hole communicated with the liquid storage bottle is disposed on the liquid collecting tank, and the via hole is higher than an inner bottom of the liquid collecting tank.

3. The fragrance diffusion equipment according to claim 2, wherein a radial length of the gap ranges from 0.20 mm to 0.40 mm;
and/or, a side wall of the liquid collecting tank is provided with a first columnar wall recessed inwards, a top side of the first columnar wall is provided with a first top plate, and the via hole is formed in the first top plate;
and/or, a diameter of via hole ranges from 0.5 mm to 1.0 mm.

4. The fragrance diffusion equipment according to claim 1, wherein the bottom end of the atomizing core penetrates through the liquid collecting tank and then extends into the liquid storage bottle, one end of the liquid suction pipe is in communication with the bottom end of the atomizing core, and the other end of the liquid suction pipe is inserted into the liquid storage bottle.

5. The fragrance diffusion equipment according to claim 1, wherein the liquid collecting tank is detachably connected to the atomizing head body, and a top edge of the liquid collecting tank is tightly matched with the atomizing head body.

6. The fragrance diffusion equipment according to claim 5, wherein the side wall of the liquid collecting tank is provided with a second columnar wall recessed inwards, a second top plate is disposed on a top side of the second columnar wall, the liquid collecting tank is fixedly connected to the atomizing head body through a bolt which penetrates from the second top plate and then is inserted into the atomizing head body, and the top edge of the liquid collecting tank is tightly matched with the atomizing head body.

7. The fragrance diffusion equipment according to claim 1, wherein an atomizing cavity is disposed in the atomizing head body, a first atomizing cover is disposed in the atomizing cavity, the first atomizing cover divides the atomizing cavity into an upper cavity and a lower cavity which are in communication with each other, and the atomizing core and the liquid collecting tank are both located in the lower cavity.

8. The fragrance diffusion equipment according to claim 7, wherein a second atomizing cover is further disposed in the lower cavity, a first atomizing channel is disposed at intervals in a circumferential direction of the first atomizing cover, the first atomizing channel is in communication with the upper cavity and the lower cavity, the second atomizing cover is located between the first atomizing cover and the atomizing core, and a second atomizing channel in communication with the lower cavity is formed between the second atomizing cover and the first atomizing cover.

9. The fragrance diffusion equipment according to claim 8, wherein the atomizing head body is provided with a gas channel in communication with the atomizing core, the gas channel is located below the second atomizing cover, a support portion is disposed at the bottom of the second atomizing cover, and the support portion abuts against a top side of the gas channel.

10. The fragrance diffusion equipment according to claim 9, wherein the second atomizing cover comprises a cover plate protruding towards the first atomizing cover, a vertical surrounding plate is disposed on the periphery of the cover plate, and a backflow gap is disposed between an outer side of the surrounding plate and the first atomizing cover; and
the first atomizing cover is provided with several pressing blocks at intervals along the periphery of a top side of the surrounding plate, the pressing blocks are matched with the support portion to vertically fix the second atomizing cover; and/or, the surrounding plate is provided with a positioning groove, the first atomizing cover is provided with a positioning plate, and the positioning plate is inserted into the positioning groove to circumferentially fix the second atomizing cover.
